(19) United States
(12) Patent Application Publication
(10) Pub. No.: US 2014/0200670 A1
(43) Pub. Date: Jul. 17, 2014

(54) SYSTEM AND METHOD FOR A SPINAL STABILIZATION IMPLANT ASSEMBLY

(71) Applicants: Kingsley R. Chin, WILTON MANORS, FL (US); Matthew IBARRA, LAKEWOOD, CA (US); Craig HENSHAW, CHARLESTOWN, MA (US); JEREMY CROSSGROVE, Storrs, CT (US); MICHAEL DRNEK, BOSTON, MA (US); LIN YIN, BROOKLINE, MA (US); AARON RICICA, BROOKLINE, MA (US)

(72) Inventors: Kingsley R. Chin, WILTON MANORS, FL (US); Matthew IBARRA, LAKEWOOD, CA (US); Craig HENSHAW, CHARLESTOWN, MA (US); JEREMY CROSSGROVE, Storrs, CT (US); MICHAEL DRNEK, BOSTON, MA (US); LIN YIN, BROOKLINE, MA (US); AARON RICICA, BROOKLINE, MA (US)

(73) Assignee: SPINEFRONTIER INC, Beverly, MA (US)

(21) Appl. No.: 13/742,898

(22) Filed: Jan. 16, 2013

(51) Int. Cl. A61F 2/44

(52) U.S. Cl. CPC A61F 2/4455
USPC 623/17.16

(57) ABSTRACT

A spinal stabilization implant assembly includes a first cervical stabilization plate comprising an elongated body having a top portion and a bottom portion, and a second cervical stabilization plate comprising an elongated body having a top portion and a bottom portion. The bottom portion of the first cervical stabilization plate is attached to a first vertebra and the top portion of the second stabilization plate is stacked end-to-end below the bottom portion of the first cervical stabilization plate and is attached to the same first vertebra. The top portion of the first cervical stabilization plate is attached to a second vertebra, and the bottom portion of the second stabilization plate is attached to a third vertebra. The second vertebra is superior to the first vertebra, and the third vertebra is inferior to the first vertebra.

### FIELD OF THE INVENTION

The present invention relates to a system and a method for a spinal stabilization implant assembly, and more particularly to a spinal stabilization implant assembly that includes a stabilization plate an intervertebral insert and bone fasteners.

### BACKGROUND OF THE INVENTION

Fibula strut grafts have a proven history of effectiveness for anterior cervical corpectomies but are inherently vulnerable to complications such as early or late fracture, dislodgement, displacement, telescoping into the vertebral body, or nonunion. The settling and resultant segmental kyphosis after multi-level anterior cervical reconstruction have also been documented. The risk of graft migration, displacement, or fracture appears more likely with more vertebral bodies removed and longer grafts, and with corpectomies involving a fusion ending at the C7 vertebral body. Newer interbody stabilization options have emerged such as polyetherketone (PEEK) which have the advantages of greater endplate coverage leading to a more stable construct and with similar modulus of elasticity as bone. However, PEEK cages require separate graft material for interbody fusion. Other options include metal expandable cages but these can be bulky, risk adjacent body fracture, and have limited room for bone graft, and therefore, do not provide the most ideal biologic environment. Stacking PEEK cages intuitively can also fill a corpectomy space by stacking the appropriate heights end-to-end. Improved cervical stabilization assemblies and methods are desirable.

### SUMMARY OF THE INVENTION

The present invention relates to a spinal stabilization implant assembly that includes a stabilization plate, an intervertebral insert and bone fasteners.

In general, in one aspect, the invention features a spinal stabilization implant assembly configured for implantation at least partially between a superior vertebra and an inferior vertebra. The spinal stabilization implant assembly includes a cervical stabilization plate and one or more bone fasteners. The cervical stabilization plate includes an elongated body having left and right side surfaces, front and back surfaces and top and bottom surfaces. The elongated body has a central portion, a top portion and a bottom portion. The top portion is bent at a first angle relative to the central portion and is dimensioned for capturing and fastening to a corner ridge of a vertebral wall of a superior vertebra. The bottom portion is bent at a second angle relative to the central portion and the second angle is opposite to the first angle and is dimensioned for capturing and fastening to a corner ridge of a vertebral wall of an inferior vertebra. The back surface has a protruding indent-tab, and the indent-tab is shaped and dimensioned to be implanted in an intervertebral space between the superior and the inferior vertebras. The elongated body further includes one or more through-openings extending from the front surface to the back surface and the one or more bone fasteners are shaped and dimensioned to be inserted through the one or more through-openings, respectively, and to be attached to locations in the vertebral walls of the superior and inferior vertebras.

Implementations of this aspect of the invention may include one or more of the following features. Each of the one or more through-openings has a first diameter at the front surface of the elongated body, a second diameter at the back surface of the elongated body and a third diameter in the area between the front the back surfaces of the elongated body. The third diameter is larger than the first diameter and the second diameter, thereby forming a lip at the top of the through-openings and a groove within an inner wall of the through openings. Each of the one or more bone fasteners includes a threaded main body and a head. The head has one or more flexible structures configured to be flexed inwards when inserted into the groove and then configured to be unflexed and to be captured within the groove. The elongated body further includes a central through opening configured to be packed with graft material. The elongated body further includes one or more spikes extending from the back surface and being shaped and dimensioned to be inserted into the vertebral walls of the superior and inferior vertebras. The spinal stabilization implant assembly further includes an intervertebral cage configured to be implanted in the intervertebral space between the superior and inferior vertebras. The intervertebral cage comprises front, back, left, right, top, and bottom surfaces. The intervertebral cage is integral with the back surface of the elongated body. The intervertebral cage further comprises first and second fins extending from back surface of the intervertebral cage. The intervertebral cage has a U-shaped body and an integrated central support bar configured to prevent movement between the superior and the inferior vertebras. The intervertebral cage is attached to the back surface of the elongated body with a screw. The top and bottom portions of the elongated body are pivotally connected to the central portion of the elongated body. The spinal stabilization implant assembly further includes top and bottom locking tabs configured to lock the angular positions of the top and bottom portions relative to the central portion. The spinal stabilization implant assembly further includes a keystone and the keystone has top and bottom angled surfaces configured to lock the angular positions of the top and bottom portions relative to the central portion, when the keystone is attached to the central portion. The cervical stabilization plate has an adjustable length. The length of the cervical stabilization plate is adjusted via a ratchet mechanism, or by rotating a threaded rod, or via a cam mechanism, or via a side-sliding mechanism. The side-sliding mechanism includes inserting plates of different height in a space between the top and central portions or the space between the bottom and central portions. The cam mechanism includes an oval shaped cam configured to be rotated in a space between the top and bottom portions. The back surface of the elongated body has a recess and the intervertebral cage is shaped and dimensioned to be inserted into the recess and to slidably engage the elongated body. The elongated body may be made of metal and the intervertebral implant may be made of PEEK. The height of the indent-tab matches the height of the intervertebral cage.

In general, in one aspect, the invention features a spinal stabilization implant assembly including a first cervical stabilization plate comprising an elongated body having a top portion and a bottom portion, and a second cervical stabilization plate comprising an elongated body having a top portion and a bottom portion. The bottom portion of the first cervical stabilization plate is configured to be attached to a first vertebra and the top portion of the second stabilization plate is configured to be stacked end-to-end below the bottom portion of the first cervical stabilization plate and to be attached to the same first vertebra. The top portion of the first cervical stabilization plate is configured to be attached to a second vertebra, and the bottom portion of the second stabilization plate is configured to be attached to a third vertebra. The second vertebra is superior to the first vertebra, and the third vertebra is inferior to the first vertebra.

In general, in one aspect, the invention features a spinal stabilization method including providing a spinal stabilization implant assembly and implanting the spinal stabilization implant assembly at least partially between a superior vertebra and an inferior vertebra. The spinal stabilization implant assembly includes a cervical stabilization plate and one or more bone fasteners. The cervical stabilization plate has an elongated body having left and right side surfaces, front and back surfaces and top and bottom surfaces. The elongated body includes a central portion, a top portion and a bottom portion. The top portion is bent at a first angle relative to the central portion and is dimensioned for capturing and fastening to a corner ridge of a vertebral wall of the superior vertebra. The bottom portion is bent at a second angle relative to the central portion and the second angle is opposite to the first angle and is dimensioned for capturing and fastening to a corner ridge of a vertebral wall of the inferior vertebra. The back surface has a protruding indent-tab. The indent-tab is shaped and dimensioned to be implanted in an intervertebral space between the superior and the inferior vertebras. The elongated body further includes one or more through-openings extending from the front surface to the back surface and the one or more bone fasteners are shaped and dimensioned to be inserted through the one or more through-openings, respectively, and to be attached to locations in the vertebral walls of the superior and inferior vertebras. The method further includes inserting an intervertebral cage in the intervertebral space between the superior and inferior vertebras, and the height of the indent-tab matches the height of the intervertebral cage.

In general, in one aspect, the invention features a spinal stabilization method including providing a first cervical stabilization plate comprising an elongated body having a top portion and a bottom portion, providing a second cervical stabilization plate comprising an elongated body having a top portion and a bottom portion, attaching the bottom portion of the first cervical stabilization plate to a first vertebra, stacking the top portion of the second stabilization plate end-to-end below the bottom portion of the first cervical stabilization plate, and attaching the top portion of the second stabilization plate to the same first vertebra. The method further includes attaching the top portion of the first cervical stabilization plate to a second vertebra, and attaching the bottom portion of the second stabilization plate to a third vertebra. The second vertebra is superior to the first vertebra, and the third vertebra is inferior to the first vertebra.

The details of one or more embodiments of the invention are set forth in the accompanying drawings and description below. Other features, objects, and advantages of the invention will be apparent from the following description of the preferred embodiments, the drawings, and the claims

### BRIEF DESCRIPTION OF THE DRAWINGS

Referring to the figures, wherein like numerals represent like parts throughout the several views:

FIG. 1A is a perspective view of two spinal stabilization assemblies stacked end-to-end on the same vertebra, according to this invention;

FIG. 1B is a perspective view of the spinal stabilization assemblies of FIG. 1A;

FIG. 2A is a perspective view of a screw used to attached the cervical stabilization plates in FIG. 1A;

FIG. 2B is a top view of the screw of FIG. 2A;

FIG. 2C is a side cross-sectional view of the installed screw of FIG. 2A;

FIG. 3 is a front view of the two spinal stabilization assemblies of FIG. 1A;

FIG. 4 is a side view of the two spinal stabilization assemblies of FIG. 1A;

FIG. 5 is a perspective view of the cervical stabilization plate in the assembly of FIG. 1A;

FIG. 6 is a front view of the cervical stabilization plate of FIG. 5;

FIG. 7 is a side view of the cervical stabilization plate of FIG. 5;

FIG. 8 is a back view of the cervical stabilization plate of FIG. 5;

FIG. 9 is a side view of another embodiment of the cervical stabilization plate of this invention;

FIG. 10 is a back view of the cervical stabilization plate of FIG. 9;

FIG. 11 is a perspective view of a combination of a cervical stabilization plate with an intervertebral insert;

FIG. 12 is a perspective view of another embodiment of a combination of a cervical stabilization plate with an intervertebral insert;

FIG. 13 is a perspective view of yet another embodiment of a combination of a cervical stabilization plate with an intervertebral insert;

FIG. 14 is a side view of the embodiment of a combination of a cervical stabilization plate with an intervertebral insert of FIG. 13;

FIG. 15A is a front view of two different size cervical stabilization plates stacked on the same vertebra;

FIG. 15B is a side cross-sectional view of the two cervical stabilization plates of FIG. 15A, along axis B-B;

FIG. 16A is a front perspective view of yet another embodiment of a spinal stabilization assembly according to this invention, including a cervical plate and an intervertebral cage; the cage is connected to the cervical plate via a screw;

FIG. 16B is a back view of the cervical stabilization plate of FIG. 16A;

FIG. 17A is a side view of a cervical plate with pivotable top and bottom portions;

FIG. 17B is another side view of the cervical plate of FIG. 17A;

FIG. 17C is a front view of the cervical plate of FIG. 17A;

FIG. 18A is a side view of another embodiment of a cervical plate with pivotable top and bottom portions;

FIG. 18B is a front view of the cervical plate of FIG. 18A;

FIG. 18C is a front perspective view of the cervical plate of FIG. 18A;

FIG. 18D is a side view of the pivotable top of the cervical plate of FIG. 18A;

FIG. 19A is a front view of a cervical plate with an adjustable length;

FIG. 19B is a front view of the cervical plate of FIG. 19A in the extended position;

FIG. 20A is a front view of another embodiment of a cervical plate with an adjustable length;

FIG. 20B is a front view of the cervical plate of FIG. 20A in the extended position;

FIG. 21A is a back perspective view of a spinal stabilization assembly according to this invention, including a cervical plate and an intervertebral cage; the cage is inserted into a recess in the back side of the cervical plate;

FIG. 21B is a back perspective view of the spinal stabilization assembly of FIG. 21A in the disassembled configuration;

FIG. 22A is a front perspective view of a cervical plate with an adjustable length via a cam mechanism;

FIG. 22B is a front view of the cervical plate of FIG. 22A in the non-extended position;

FIG. 22C is a front view of the cervical plate of FIG. 22A in the extended position;

FIG. 23A is a side view of a cervical plate with pivotable top and bottom portions; the pivot angle is set and locked with a keystone;

FIG. 23B is a front perspective view of the cervical plate of FIG. 23A with different keystones for setting different pivot angles;

FIG. 23C is a side view of the cervical plate of FIG. 23A;

FIG. 24A is a front view of another embodiment of a cervical plate with an adjustable length;

FIG. 24B is a perspective view of the cervical plate of FIG. 24A in the expanded configuration;

FIG. 24C is a detailed view of the sliding expansion mechanism of the cervical plate of FIG. 24A.

FIG. 25A is a front view of yet another embodiment of the stabilization assembly of this invention having a cervical plate with an adjustable length;

FIG. 25B is a perspective view of the stabilization assembly of FIG. 25A in the expanded configuration;

FIG. 25C is a front view of an expanded configuration of the assembly of FIG. 25A;

FIG. 25D is a front view of a non-expanded configuration of the assembly of FIG. 25A;

FIG. 25E is a front perspective view of an expanded configuration of the assembly of FIG. 25A;

FIG. 26A-FIG. 26D depict yet another embodiment of the stabilization assembly of this invention having a cervical plate with pivotable top and bottom portions;

FIG. 27A depicts the step of inserting the intervertebral cage in the disc space;

FIG. 27B depicts the step of drilling holes with the guidance of a drill guide;

FIG. 27C depicts the step of securing the cervical plate onto the vertebral body and the cage via screws; and

FIG. 27D depicts the assembled stabilization implant.

### DETAILED DESCRIPTION OF THE INVENTION

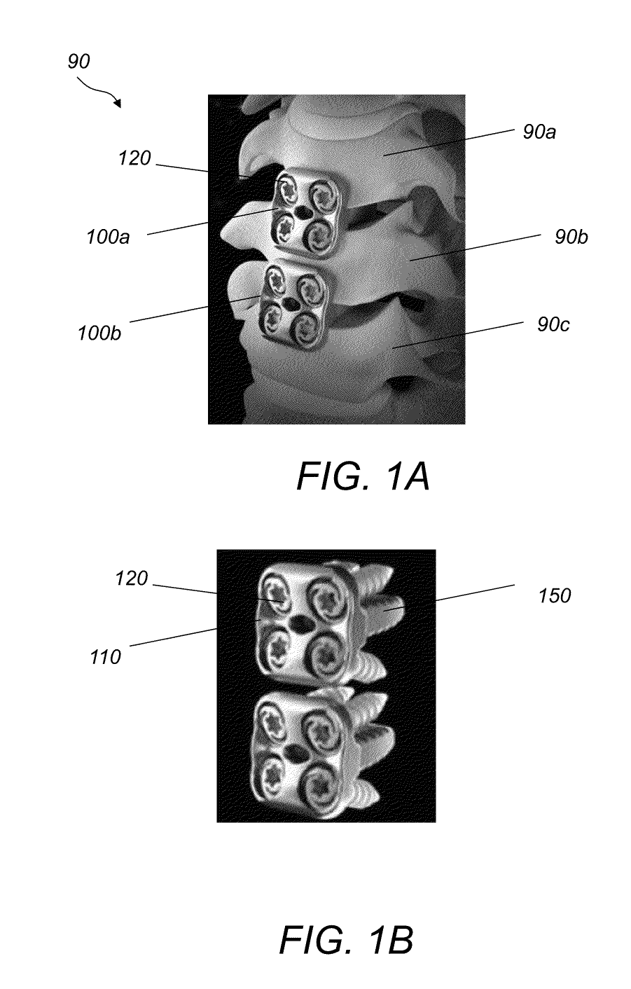

The present invention relates to a spinal stabilization implant assembly 100 that includes a cervical stabilization plate 110, an intervertebral cage insert 150 and one or more bone fasteners 120, as shown in FIG. 1A and FIG. 1B.

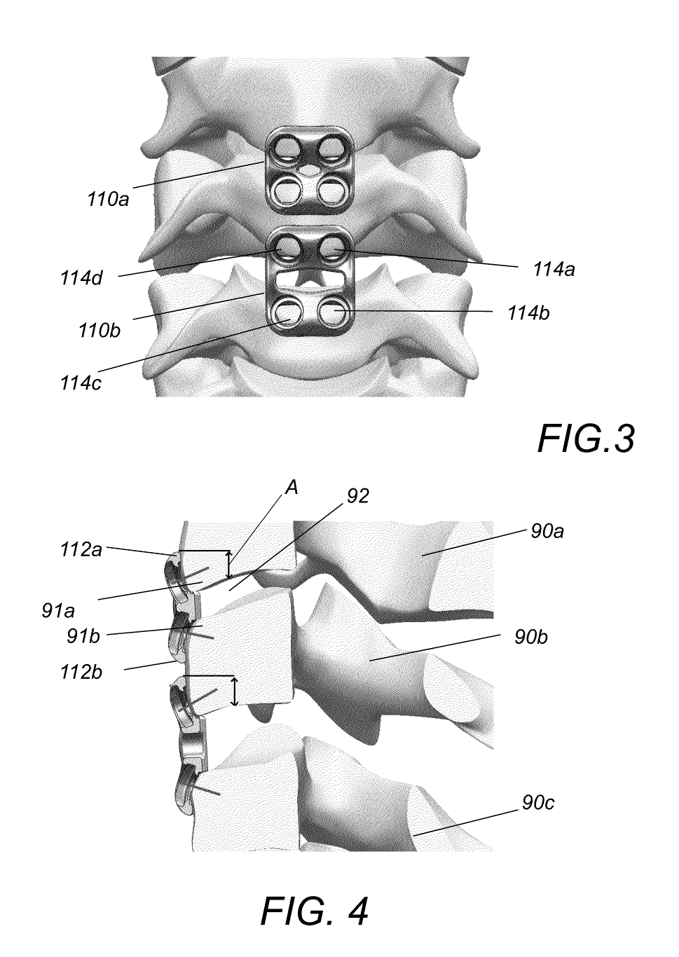

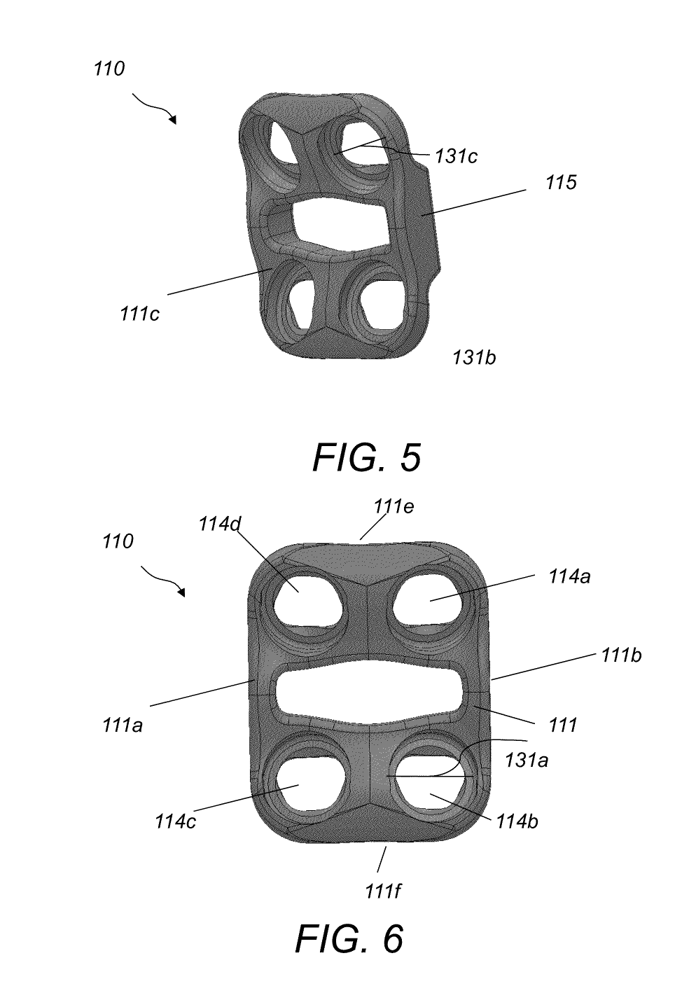

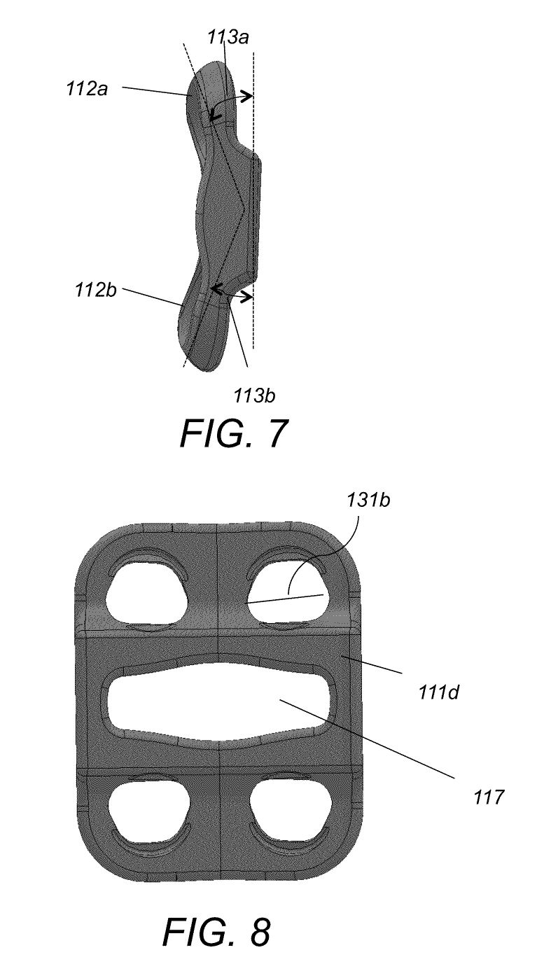

Referring to FIG. 3 to FIG. 8, the cervical plate 110 includes an elongated body 111 having left and right side surfaces 111a, 111b, front and backs surfaces 111c, 111d, and top and bottom surfaces 111e, 111f. The elongated body 111 includes one or more through-openings 114a, 114b, 114c, 114d extending from the front surface to the back surface at the top 112a and bottom 112b portions of the elongated body 111. Elongated body 111 also includes a central opening 117 configured to hold bone growth material. The top portion 112a is bent at an angle 113a ideal for capturing and fastening to the corner ridge 91a of the vertebral wall, as shown in FIG. 4. The bottom portion 112b is bent at an opposite angle 113b also ideal for capturing and fastening to the corner ridge 91b of the lower vertebral wall. The back surface 111d includes a protruding indent-tab 115 designed to fit in the intervertebral space 92. The elongated body 111 has variable lengths that vary only in the middle portion extending the indent-tab. The height of the indent-tab varies based on the height of the corresponding intervertebral body used in conjunction with this plate for vertebral fusion and fixation.

The one or more bone fasteners 120 are configured to be inserted through the one or more through-openings 114a, 114b, respectively, and to be attached to locations in the spinal vertebras, thereby attaching the cervical plate to the spinal vertebras. The through-openings comprise a first diameter 131a at the front surface 111c of the elongated body 111, a second diameter 131b at the back surface 111d of the elongated body 111 and a third diameter 131c in the area between the front 111c and back 111d surfaces of the elongated body 111. The first diameter 131a is smaller than the third diameter 131c, thereby forming a lip 132 at the top of the through-openings. The third diameter 131c is larger than the second diameter 131b and the first diameter 131a is larger than the second diameter 131b, thereby forming a groove 133 within the inner wall of the through-openings. The bone fasteners 120 comprise a threaded main body 124 and a head 122. The threaded main body 124 comprises threads 124a for engaging the spinal vertebras and the head 122 comprises one or more flexible structures 121a, 121b, 121c configured to be flexed and inserted into the groove 133 and then unflex and remain captured within the groove 133.

Among the advantages of the invention may be one or more of the following. The spinal stabilization implant assembly has ultra-low profile design, dual integrated screw lock mechanisms that prevent screw backout, pre-angled zero-step lock with variable screws, large graft window for visibility and anterior graft packing, tactile, audible and visual feedback of screw engagement, 20° variable screw angulation, and safe and simple screw recovery feature, among others. PEEK cages may be stacked around a fibula strut graft to fill a corpectomy defect. This practice may limit the chance of endplate fracture or graft dislodgment. There is space around the fibula graft for additional osteobiologics such as demineralized bone matrix (DBM). There may be benefit to designing PEEK cages that are stackable.

The method of implanting the spinal stabilization implant assembly of this invention includes the following. After determining cage size and inserting the cage in place, a void in the annulus remains. Next, place drill guide over annulus void. Indent of drill guide will fit through, thereby showing ideal location for plate placement and hole location on the vertebrae. Next, drilling through holes of drill guide and then removing the drill guide. Next, grabbing the plate with the plate holder and placing it over annulus void and screw holes. The plate size is adjusted to correspond to the cage size used in disc space. The indent of plate is selected to fit in the annulus void. Next, inserting screws to secure plate to the vertebral body. If performing multiple level fixation/fusion, repeat steps at next level. Each assembly includes a cervical plate, a cervical cage, a drill guide and a plate holder

FIG. 1A depicts a perspective view of two spinal stabilization assemblies 100a, 100b stacked end-to-end on the same vertebra 90b, according to this invention. Each assembly includes a cervical plate 110, an intervertebral cage 150 and four fastening screws 120 for attaching the cervical plate to the vertebra, as shown in FIG. 1B.

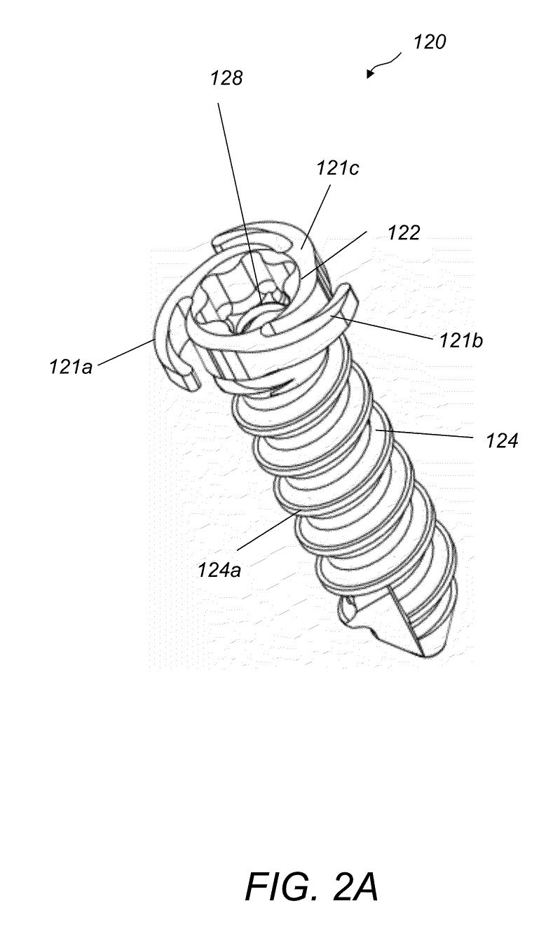

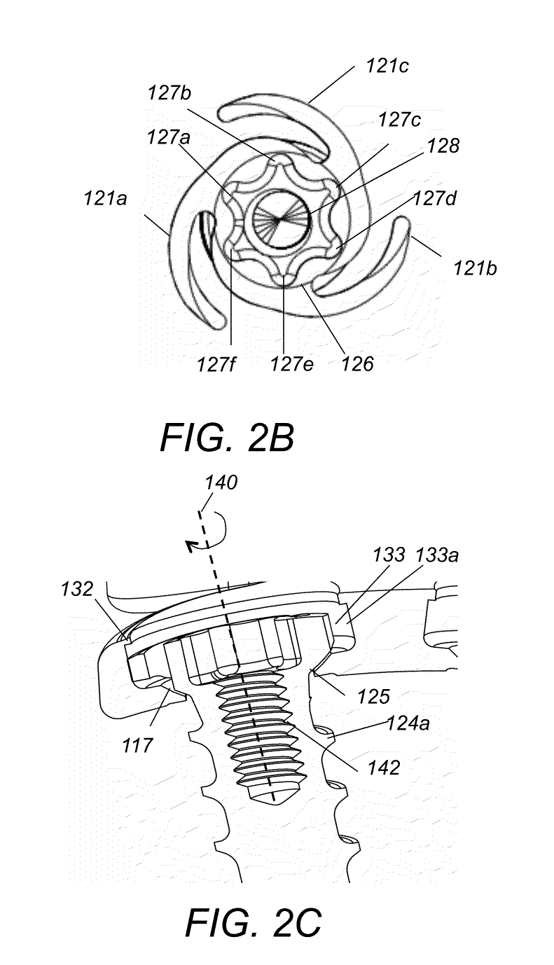

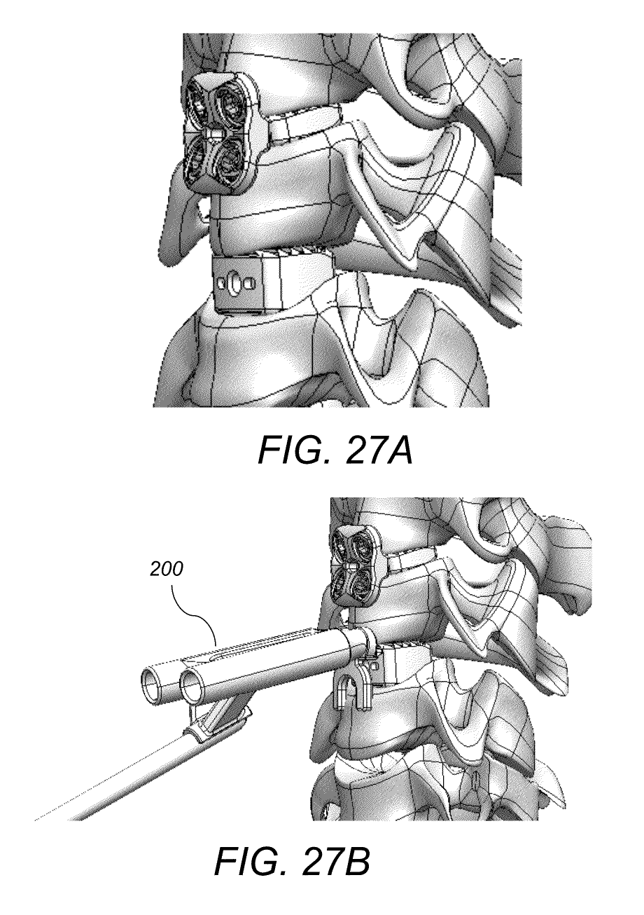

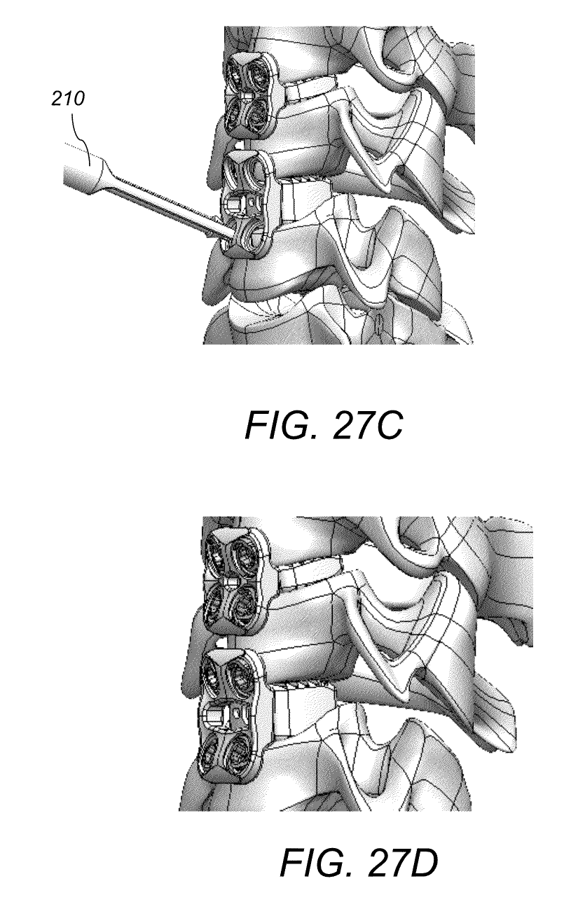

Referring to FIG. 2A, FIG. 2B, and FIG. 2C, bone screw 120 has a threaded main body 124 and a head 122. Main body 124 includes threads 124a for engaging the vertebral bone. Head 122 has a flat top 123, a cylindrical center 126 and a tapered portion 125 with angled bottom sides. Top 123 includes an opening 128 extending into the main body 124. Opening 128 has six lobes 127a-127f, and the geometry of opening 128 interfaces with the geometry of a screw engaging component 284 to lock a driver tool 200 into the opening 128, as shown in FIG. 27C. Three flexible arms 121a-121c extend tangentially from the outer side of the cylindrical center 126 and curve around the center 126. The effective diameter of the screw head 122 including the arms 121a-121c in the unflexed position is larger than the top diameter 131a of openings 114a-114d, shown in FIG. 6. Arms 121a-121c flex inward toward the central axis 140 when they come in contact with lip 132 of the openings 114a-114d while the screw 120 is being rotated clock-wise to be driven into the vertebral body. The effective diameter of the screw head 122 including the arms 121a-121c in the inward flexed position is smaller than the top diameter 131a of openings 114a-114d, and this allows the screw head 122 including the arms 121a-121c to move below the lip 132. Once the arms 121a-121c are below the lip 132 they expand back up to their unflexed position within the space (groove) 133 formed in the opening 114a between the lip 132 and the chamfered sides at the bottom portion 117 of the opening. Once the entire screw head 122 is in place within space 133, the lip 132 prevents the screw head from accidentally moving up (i.e., backing out) from space 133 due to stresses applied during spinal motion. In cases where the mounted screw is rotated counter-clockwise, arms 121a-121c hit the lip 132 and sidewall 133a and flex outward away from the central axis 140, thereby increasing the effective diameter of the screw head so that it is even larger than the top diameter 131a. This outward flexing of the arms 121a-121c prevents the screw head 122 from accidentally moving up and out of space 133. The surgeon may pull out the screw with a driver tool 200, as shown in FIG. 27C.

FIG. 3 also depicts two spinal stabilization assemblies stacked end-to-end on the same vertebra. These assemblies include two different size cervical plates 110a, 110b. As was mentioned above, the cervical plate recesses into the disc space 92 and hugs the edge of the vertebral endplate, so regardless of the disc space 92, the edge of the cervical plate with respect to the vertebral endplate (distance A) is always the same, and therefore two cervical plates can be stacked end-to-end on the same vertebra, as shown in FIG. 4.

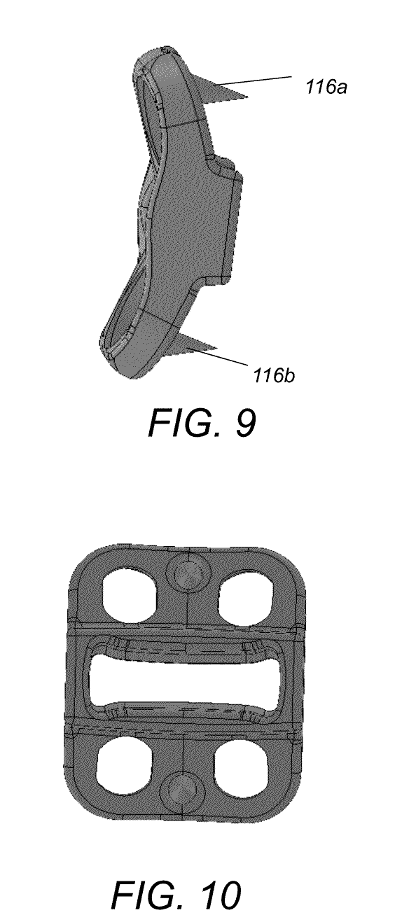

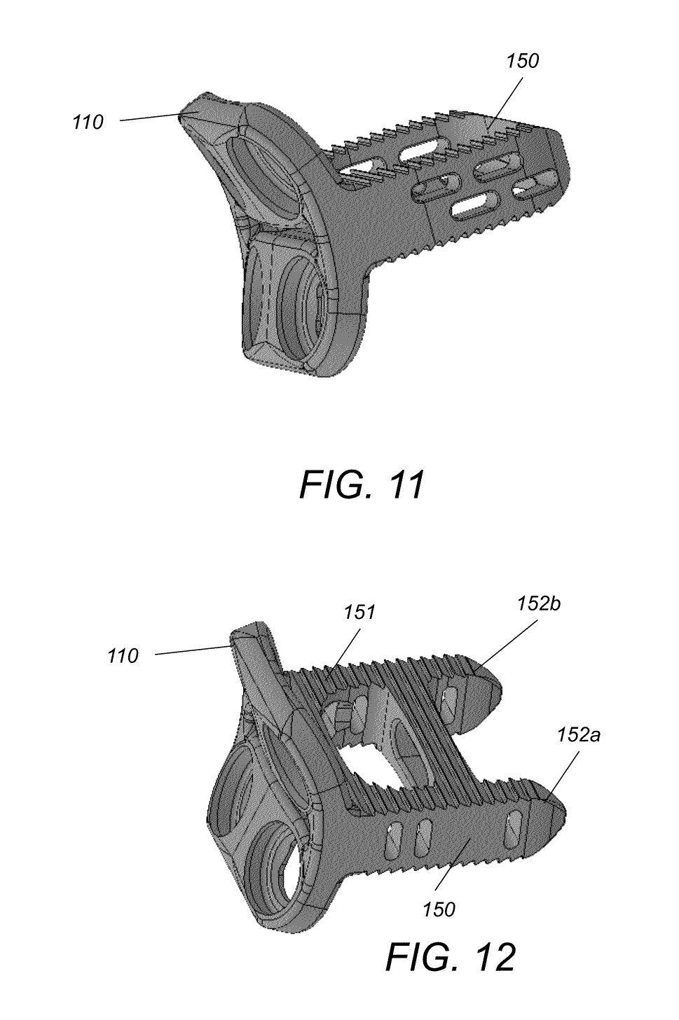

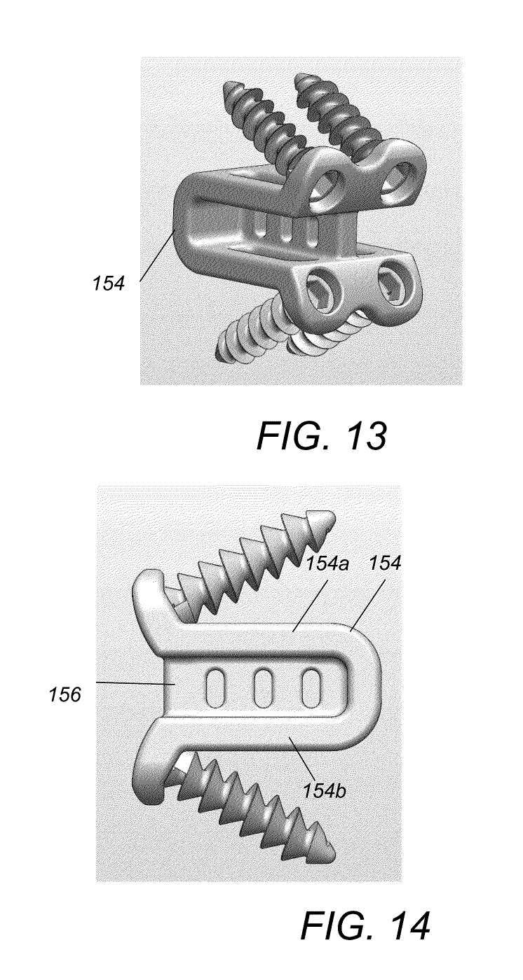

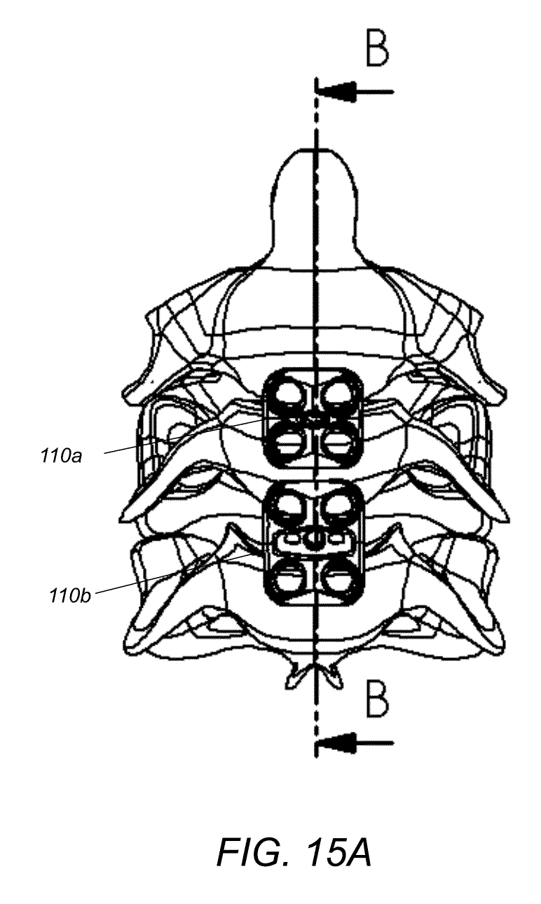

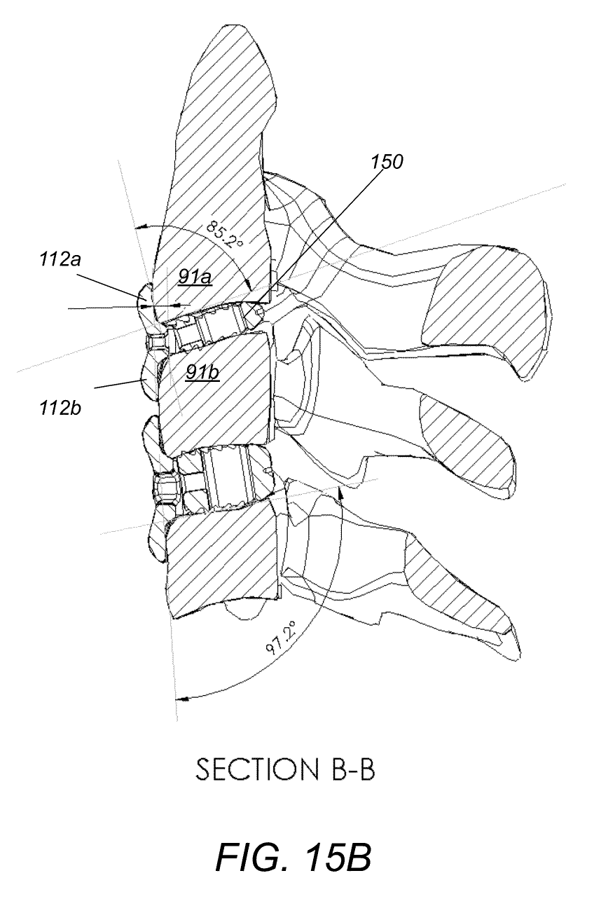

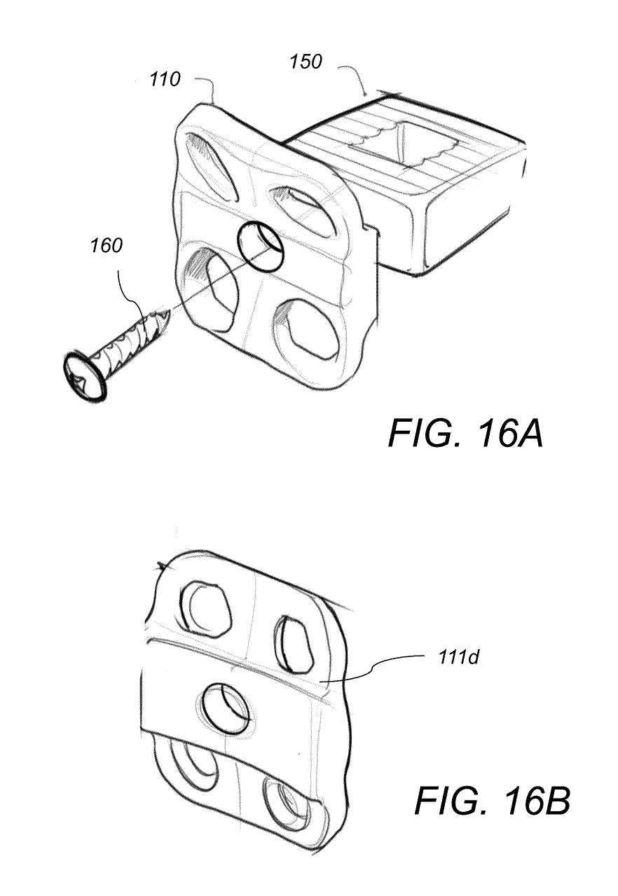

FIG. 9 is a side view of another embodiment of the cervical stabilization plate of this invention. In this embodiment the back side of the cervical plate includes spikes 116a, 116b that are inserted into the vertebral bodies. FIG. 11 is a perspective view of a combination of a cervical stabilization plate 110 with an intervertebral cage 150. In this embodiment the cervical plate 110 is integrated with the intervertebral cage 150 and the back of the cage is closed. FIG. 12 is a perspective view of another embodiment of a combination of a cervical stabilization plate 110 with an intervertebral cage 150. In this embodiment the cervical plate is integrated with the intervertebral cage and the back of the cage 150 is open and has two fins 152a, 152b. The top surfaces of the cage include ridges 151. FIG. 13 is a perspective view of yet another embodiment of a combination of a cervical stabilization plate 110 with an intervertebral cage 150. In this embodiment, the intervertebral cage 150 has a U-shape body and a central bar 156 connecting the top and bottom components 154a, 154b of the U-shaped body. Bar 156 is integral to the U-shaped body and is configured to prevent movement between the superior and the inferior vertebras. FIG. 16A is a front perspective view of yet another embodiment of a spinal stabilization assembly according to this invention, including a cervical plate 110 and an intervertebral cage 150. The cage is separate from the cervical plate 110 and is connected to the cervical plate via a screw 160.

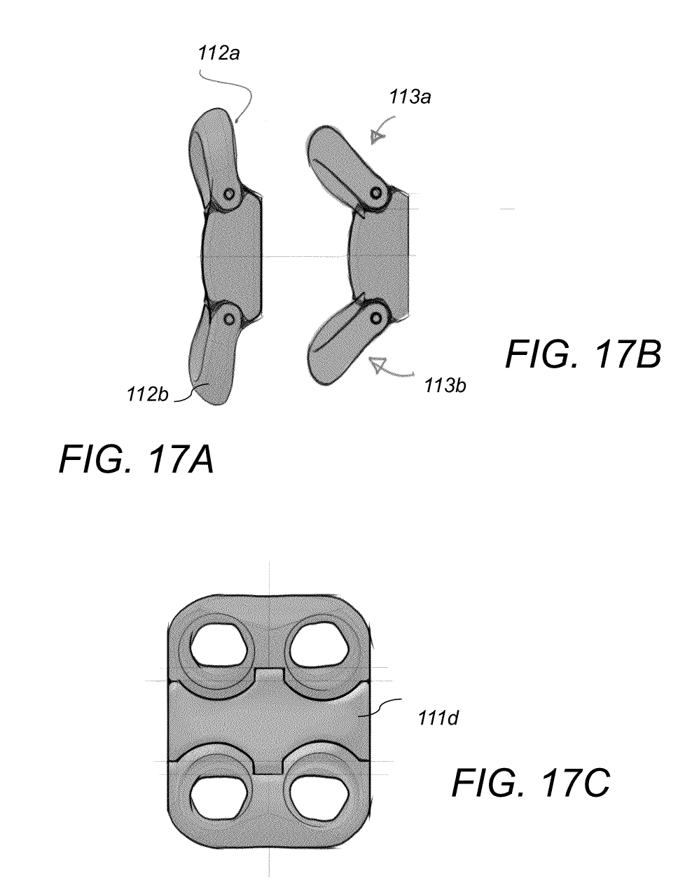

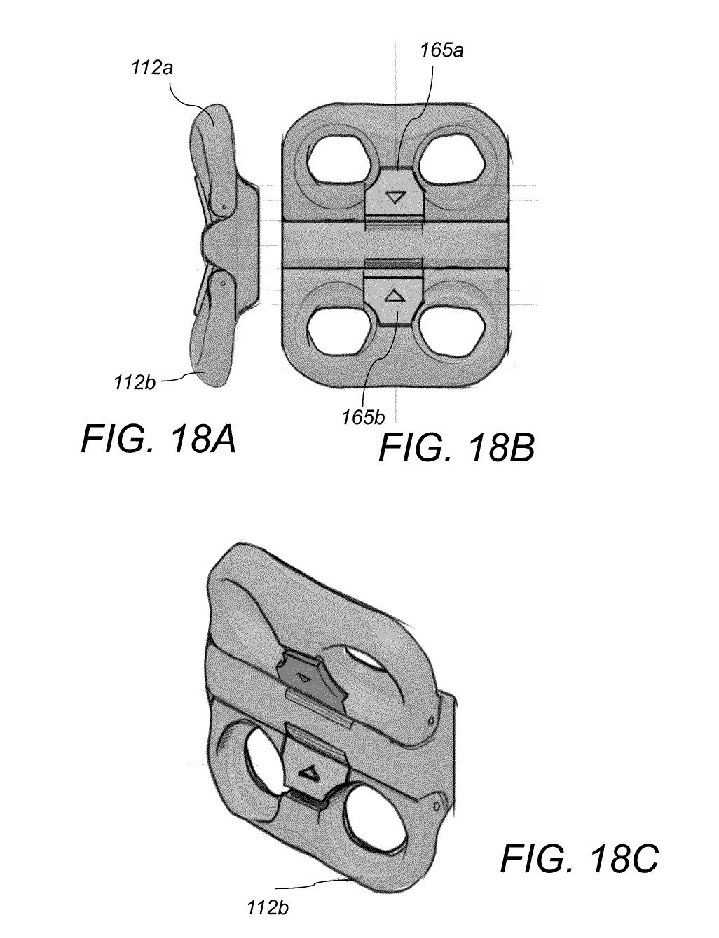

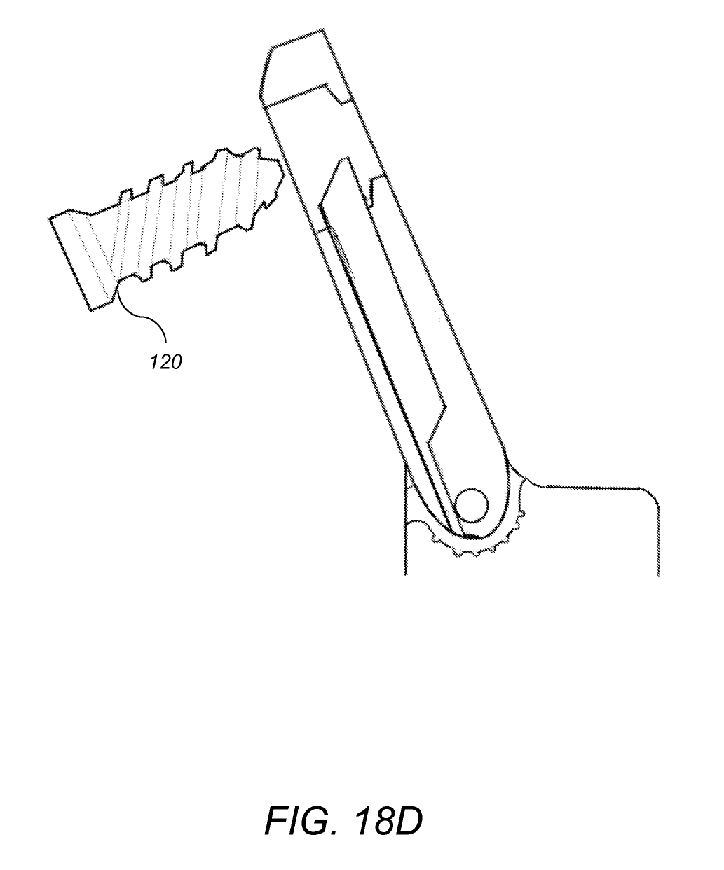

FIG. 17A is a side view of a cervical plate 110 with pivotable top and bottom portions 112a, 112b. In this embodiment the pivotable top and bottom portions 112a, 112b are non-locking. The angle 113a relative to the main portion of the elongated body is set and the fastening screws 120 secure the set angle when fastened. FIG. 18A is a side view of another embodiment of a cervical plate 110 with pivotable top and bottom portions 112a, 112b. In this embodiment the pivot angles 113a, 113b of the pivotable top and bottom portions are locked. The pivot angles relative to the main portion of the elongated body are set and the front tabs 165a, 165b are pressed and locked via the fastening screws 120, as shown in FIG. 18D.

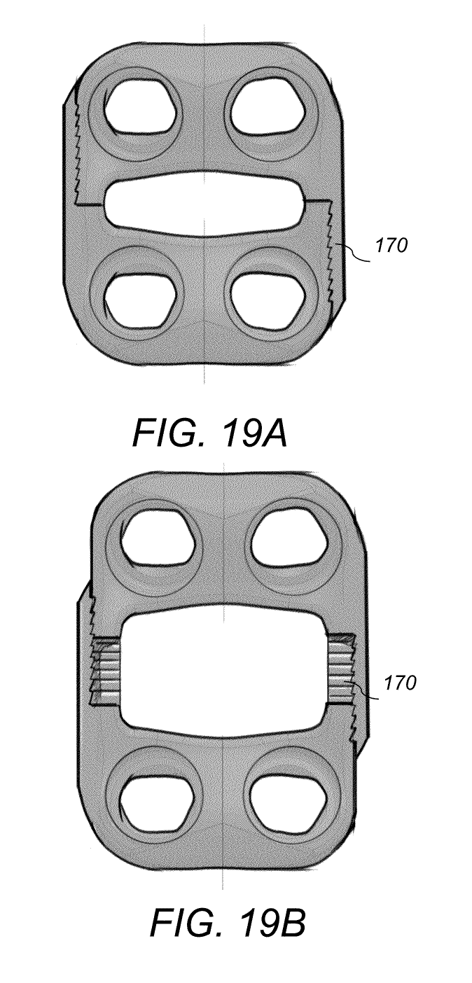

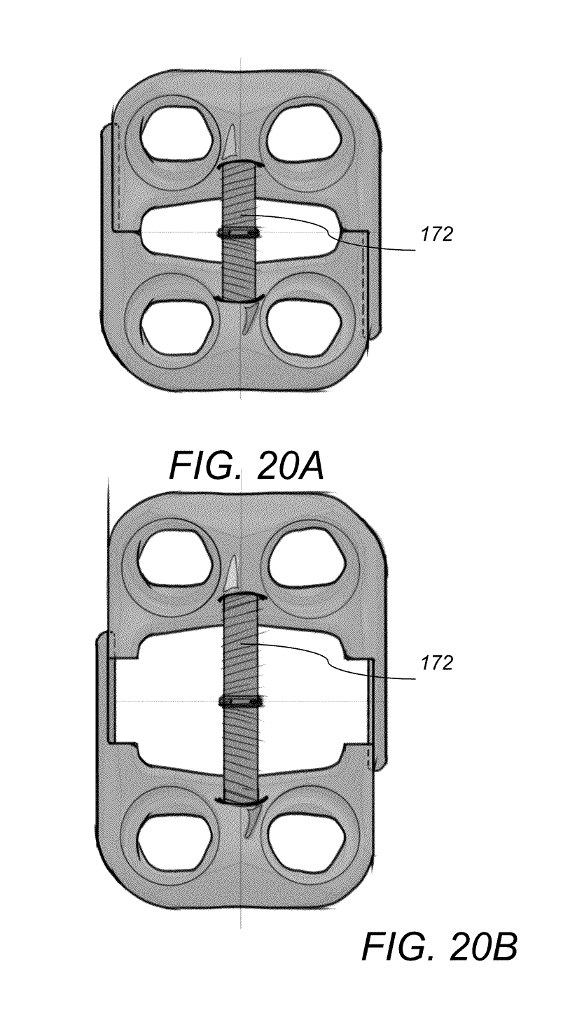

FIG. 19A is a front view of a cervical plate with an adjustable length. The length is adjusted via a ratchet mechanism 170, as shown in FIG. 19B. FIG. 20A is a front view of another embodiment of a cervical plate with an adjustable length. The length is adjusted by rotating clockwise or counter-clockwise a threaded rod 172, as shown in FIG. 20B.

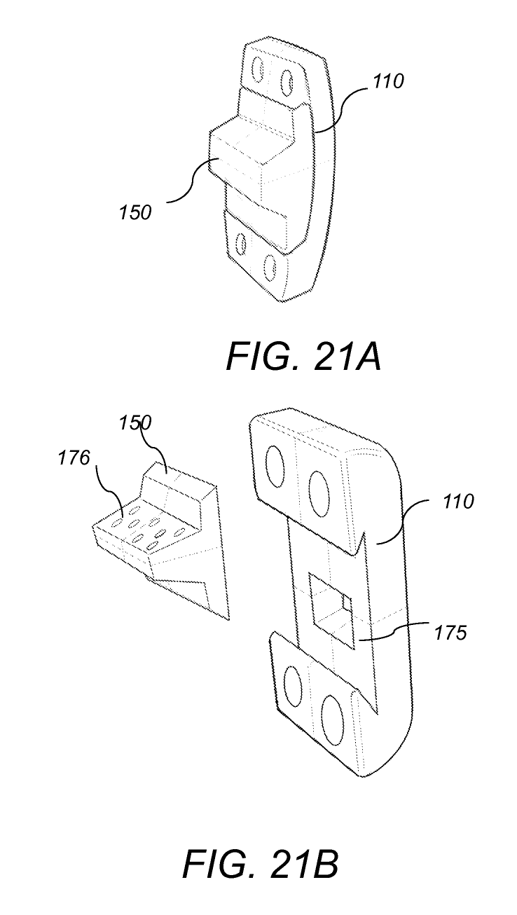

FIG. 21A is a back perspective view of a spinal stabilization assembly 100 according to this invention, including a cervical plate 110 and an intervertebral cage 150. The cage 150 is inserted into a recess in the back side of the cervical plate 110. The cage is made of PEEK and the plate is made of Ti metal. The holes in the cage 176 are used for holding blood and bone ingrowth.

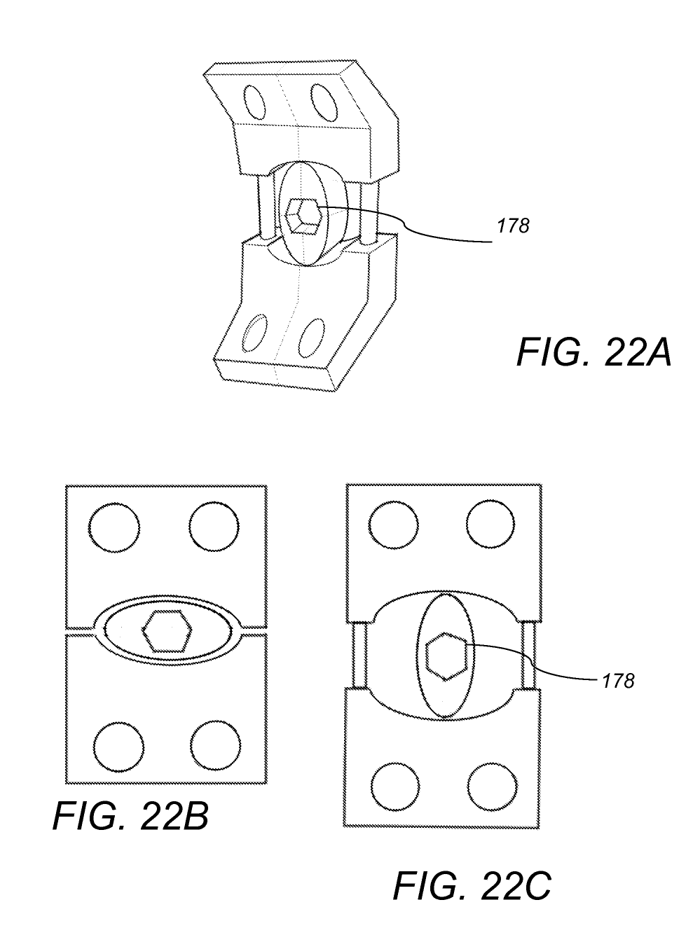

FIG. 22A is a front perspective view of a cervical plate with an adjustable length. In this embodiment the plate length is adjusted via a cam mechanism 178. FIG. 22B is a front view of the cervical plate of FIG. 22A in the non-extended position and FIG. 22C is a front view of the cervical plate of FIG. 22A in the extended position. The center cam 178 is twisted to expand the plate.

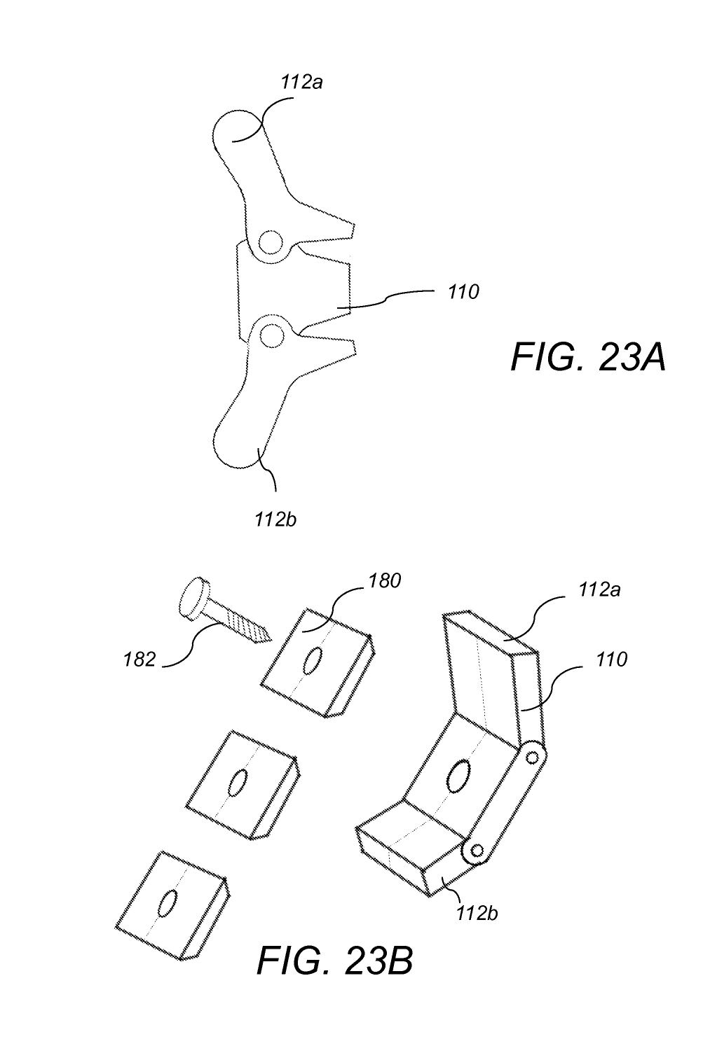

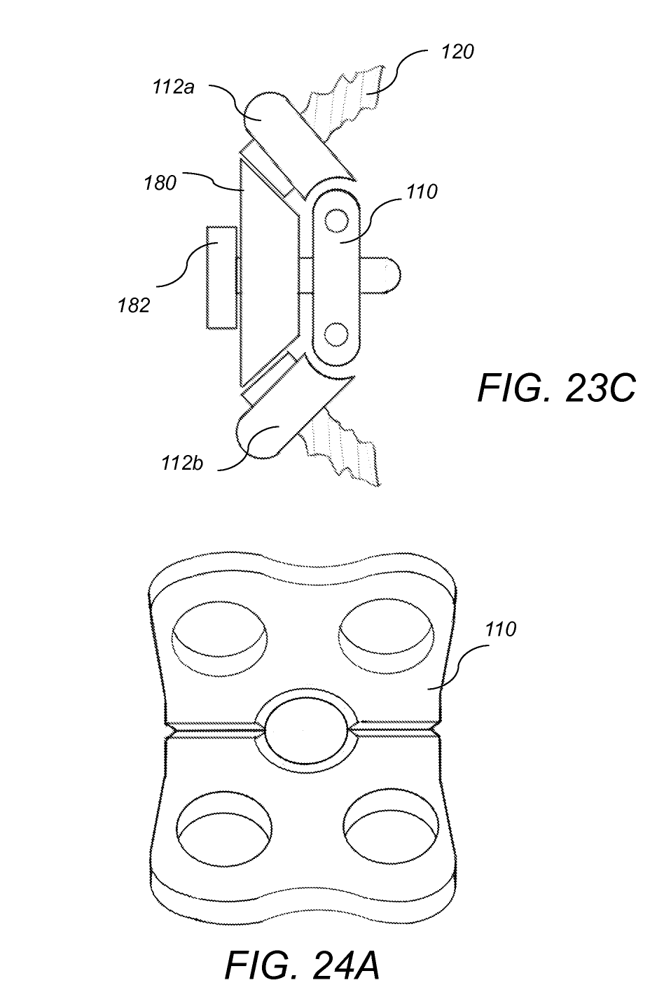

FIG. 23A is a side view of a cervical plate 110 with pivotable top and bottom portions 112a, 112b. The pivot angles 113a, 113b are set and locked with a keystone 180. Different keystones are used for setting different pivot angles, as shown in FIG. 23B. The keystone block 180 is secured with screw 182 and locks in all other fastening screws 120, as shown in FIG. 23C.

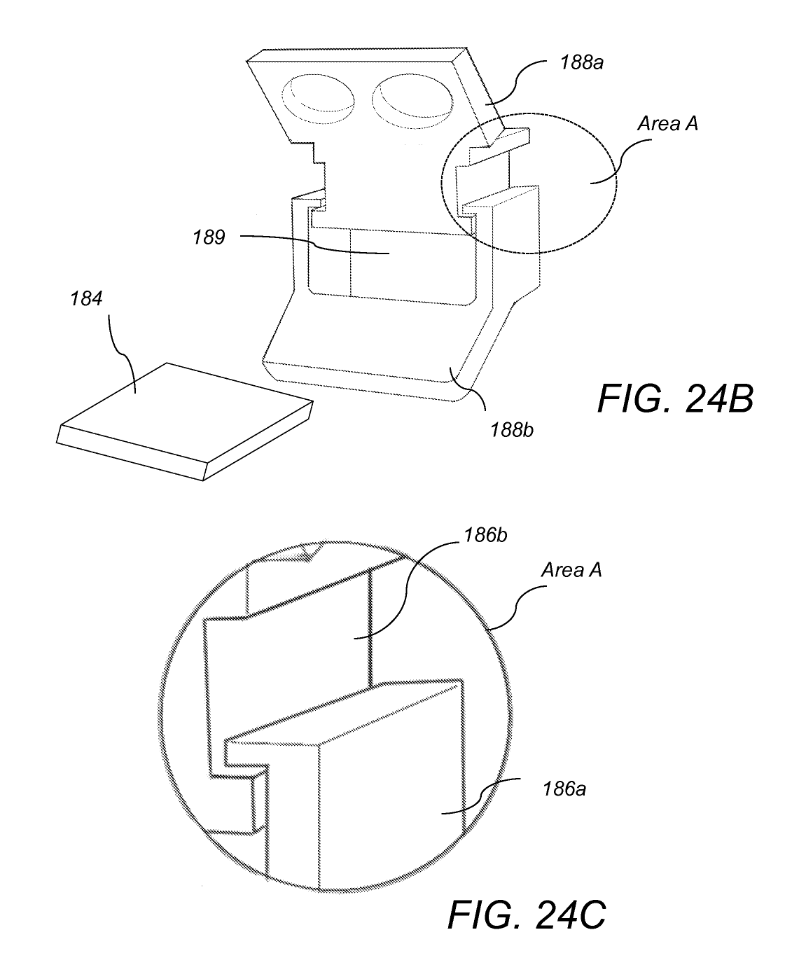

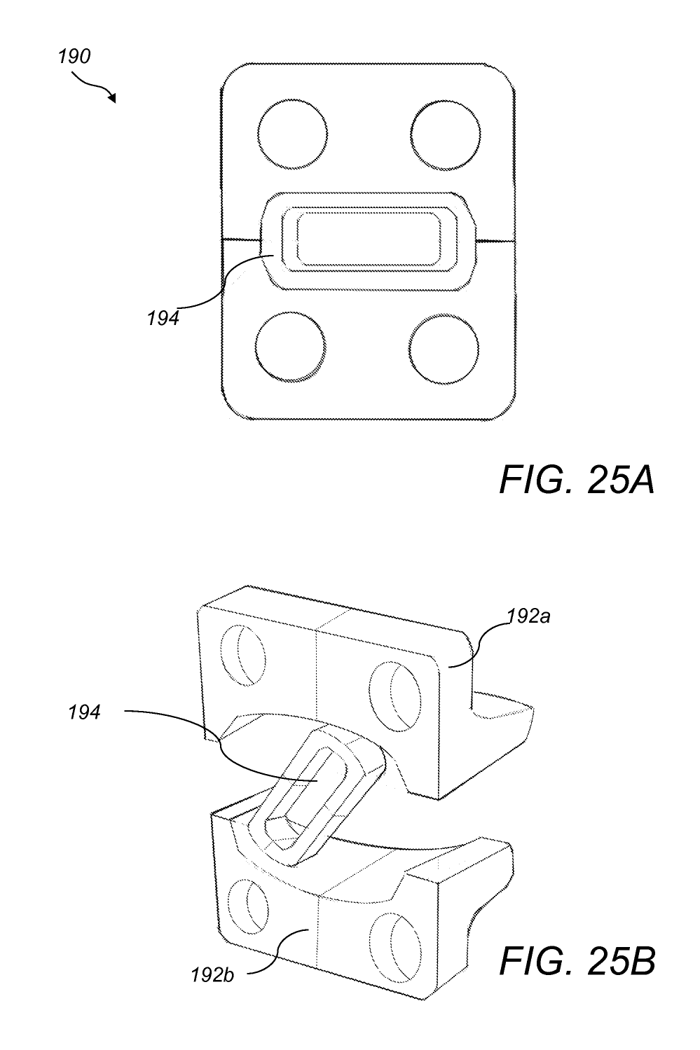

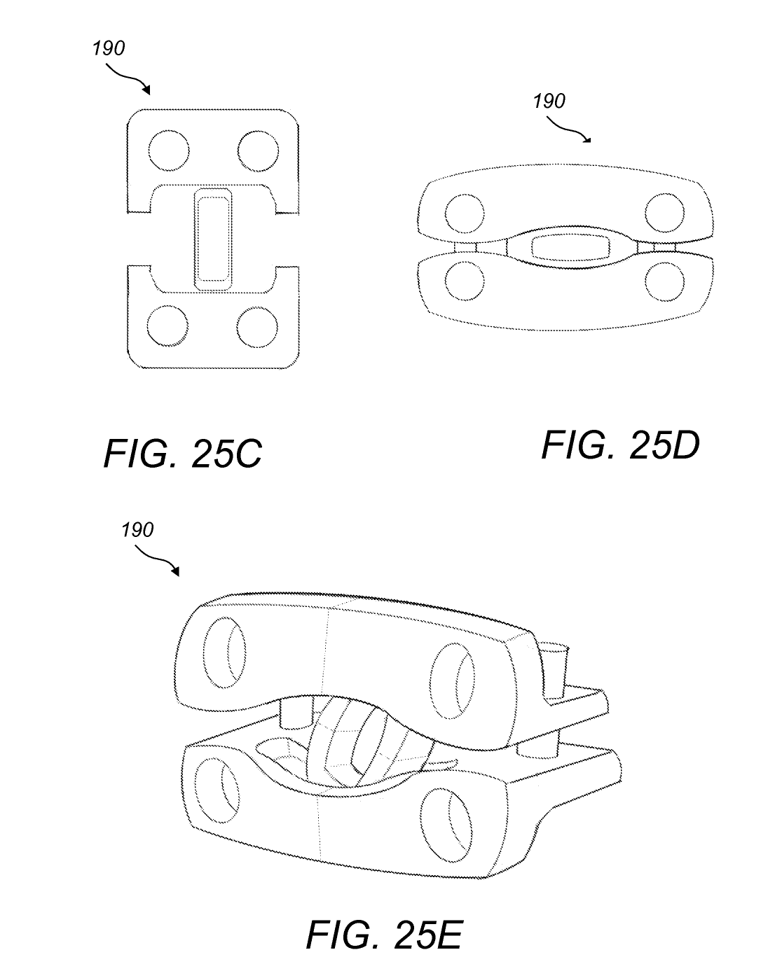

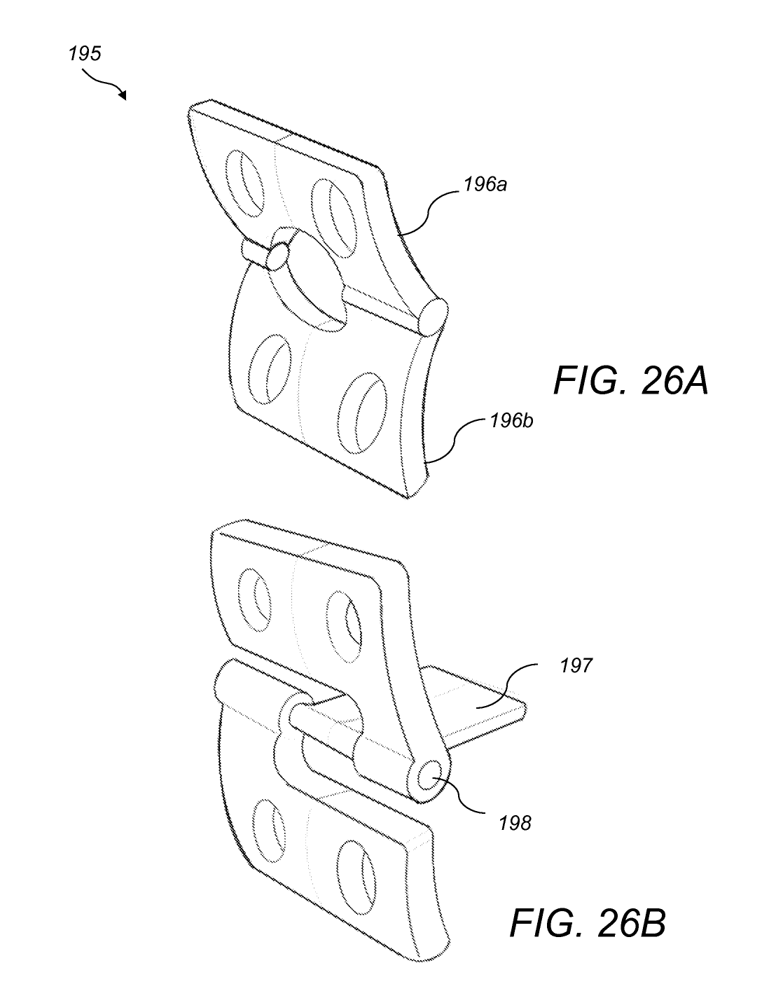

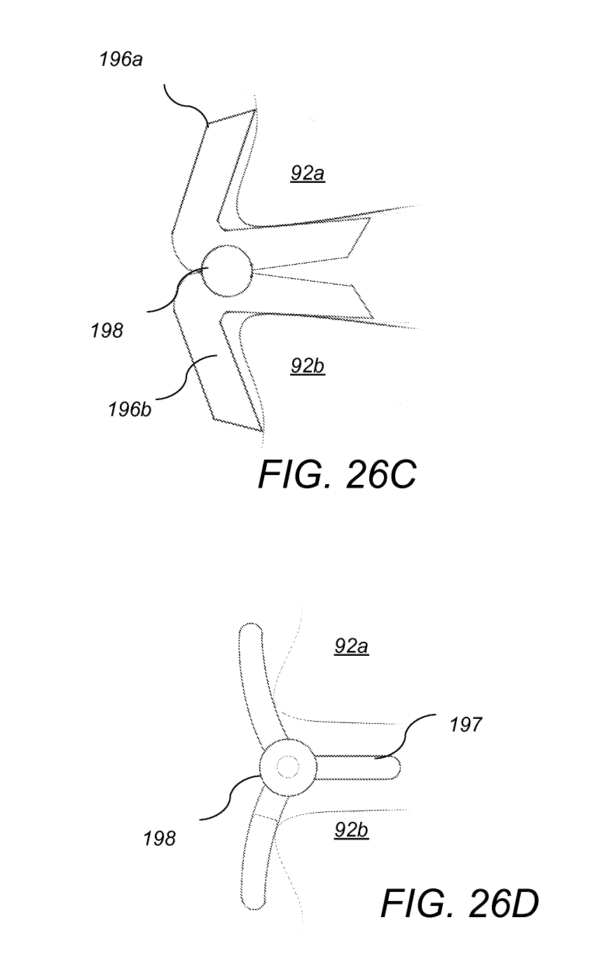

FIG. 24A is a front view of another embodiment of a cervical plate with an adjustable length. In this embodiment, the length is adjusted via a side-sliding mechanism, shown in FIG. 24B and FIG. 24C. Sides 186a, 186b are slidably connected and the length of plate 110 is adjusted by sliding the top component 188a relative to the bottom component 188b and then inserting a plate 184 in the opening 189 formed between the top and bottom components 188a, 188b. FIG. 25A is a front view of yet another embodiment of the stabilization assembly of this invention having a cervical plate with an adjustable length. The cervical plate includes top and bottom portions 192, 192b. An oval shaped cam 194 rotates in the center of the plate to spread the top and bottom portions 192a, 192b of the plate apart and to bring them together, as shown in FIG. 25B-FIG. 25E. FIG. 26A-FIG. 26D depict yet another embodiment of the stabilization assembly of this invention having a cervical plate 195 with pivotable top and bottom portions 196a, 196b. Top and bottom portions 196a, 196b are pivotally connected via a pivot pin 198. The intervertebral insert may be part of the pivotable top and bottom portions 196a, 196b, as shown in FIG. 26C. In other embodiments, the intervertebral insert is a separate pivotable component 197, shown in FIG. 26B and FIG. 26D.

FIG. 27A depicts the step of inserting the intervertebral cage in the disc space. FIG. 27B depicts the step of drilling holes into the vertebral bodies with the guidance of a drill guide 200. FIG. 27C depicts the step of securing the cervical plate 110 onto the vertebral body and the cage by attaching with screws 120 with a driver 210. FIG. 27D depicts the assembled stabilization implant.

The present invention also provides a new approach to filling a corpectomy defect by stacking multiple PEEK cages around a fibula strut graft. The method has been used to treat a 45 year old male who underwent C5 corpectomy with a fibula strut allograft inside three PEEK cages stacked vertically around the graft. Follow-up at nine months showed improved strength, the patient returning to regular daily activities, and cervical spine x-rays demonstrating radiographic evidence of graft consolidation consistent with fusion. There was no evidence of subsidience or focal kyphosis.

In another case, the method was use to treat a 47 year old man with a massive C5-6 herniated disc. This patient's cervical radiculomyelopathy was attributed to the C5-6 herniated disc. The patient underwent anterior cervical decompression and fusion (ACDF). A 7 mm PEEK interbody cage (Invibio PEEK-OPTIMA) and a 19 mm cervical plate and screws (SpineFrontier Inc, Indus Invue Plate, Beverly, Mass.) were used to stabilize the spine. At six weeks postoperatively the patient still had residual pain and bilateral arm numbness. A repeat MRI illustrated improvement but computed tomography (CT) showed large posterior osteophytes causing residual stenosis at the level of the C5 and C6 endplates along with the functional compression at C3-C4. The patient underwent revision C5 corpectomy and a C3-4 ACDF. An 8 mm cage (Eminent Spine, Texas) with autograft corpectomized bone and DBM were placed at C3-4. Distraction pins were placed in the body of C4 and C6 and we measured for a 30 mm long fibula strut graft. A fibula strut allograft was fashioned and cut in half length wise and placed through three PEEK cages for a total length of 30 mm (12 mm, 10 mm, 8 mm in this order). The combined cages and fibula strut graft were placed in the trough and the distraction pins removed to allow the C4 and C6 vertebrae to collapse around the construct. Demineralized bone matrix (DBM) and autograft corpectomized bone were placed within the cages alongside the fibula strut graft. A 60 mm cervical plate with lordosis was placed with screws (SpineFrontier Inc, Indus Invue Plate, Beverly, Mass.). This plate felt solidly fixed and DBM was placed behind the plate and in front of the cages at all levels. At one year he had mild residual complaints of neck pain, numbness and tingling but was much improved overall. CT showed evidence of graft consolidation consistent with fusion. The plate and screws were stably fixed.

Although fibula strut grafts are historically an effective option for anterior cervical corpectomy, they are vulnerable to complications as the number of levels decompressed increases. Associated donor site morbidity is an additional consideration. Patients experiencing compliance difficulties with cervical bracing when fibula strut grafts are used without plating or with buttress plating may be at increased risk of graft failure. Patients with a history of heavy narcotic use or smoking may be at further risk for unfavorable outcomes without a stable fibula strut graft after corpectomy. In contrast, PEEK cages have good biomechanical characteristics and a comparable elastic coefficient to that of human bone. ACDF with a PEEK cage has shown good clinical results for single level cervical disorders, but this is not the case with multi-level ACDF.

Several embodiments of the present invention have been described. Nevertheless, it will be understood that various modifications may be made without departing from the spirit and scope of the invention. Accordingly, other embodiments are within the scope of the following claims.

What is claimed is:

1. A spinal stabilization implant assembly configured for implantation at least partially between a superior vertebra and an inferior vertebra comprising:
a cervical stabilization plate comprising an elongated body having left and right side surfaces, front and back surfaces and top and bottom surfaces;
one or more bone fasteners;
wherein the elongated body comprises a central portion, a top portion and a bottom portion and wherein the top portion is bent at a first angle relative to the central portion and is dimensioned for capturing and fastening to a corner ridge of a vertebral wall of a superior vertebra and wherein the bottom portion is bent at a second angle relative to the central portion and wherein the second angle is opposite to the first angle and is dimensioned for capturing and fastening to a corner ridge of a vertebral wall of an inferior vertebra;
wherein the back surface comprises a protruding indent-tab, and wherein the protruding indent-tab is shaped and dimensioned to be implanted in an intervertebral space between the superior and the inferior vertebras; and
wherein the elongated body further comprises one or more through-openings extending from the front surface to the back surface and wherein the one or more bone fasteners are shaped and dimensioned to be inserted through the one or more through-openings, respectively, and to be attached to locations in said vertebral walls of the superior and inferior vertebras.

2. The spinal stabilization implant assembly of claim 1, wherein each of the one or more through-openings comprises a first diameter at the front surface of the elongated body, a second diameter at the back surface of the elongated body and a third diameter in the area between the front the back surfaces of the elongated body and wherein the third diameter is larger than the first diameter and the second diameter, thereby forming a lip at the top of the through-openings and a groove within an inner wall of the through openings.

3. The spinal stabilization implant assembly of claim 2, wherein each of the one or more bone fasteners comprises a threaded main body and a head and wherein the head comprises one or more flexible structures configured to be flexed inwards when inserted into the groove and then configured to be unflexed and to be captured within the groove.

4. The spinal stabilization implant assembly of claim 1, wherein the elongated body further comprises a central through opening configured to be packed with graft material.

5. The spinal stabilization implant assembly of claim 1, wherein the elongated body further comprises one or more spikes extending from the back surface and being shaped and dimensioned to be inserted into the vertebral walls of the superior and inferior vertebras.

6. The spinal stabilization implant assembly of claim 1, further comprising an intervertebral cage configured to be implanted in the intervertebral space between the superior and inferior vertebras, and wherein the intervertebral cage comprises front, back, left, right, top and bottom surfaces.

7. The spinal stabilization implant assembly of claim 6, wherein the intervertebral cage is integral with the back surface of the elongated body.

8. The spinal stabilization implant assembly of claim 6, wherein the intervertebral cage further comprises first and second fins extending from back surface of the intervertebral cage.

9. The spinal stabilization implant assembly of claim 7, wherein the intervertebral cage comprises a U-shaped body and an integrated central support bar configured to prevent movement between the superior and the inferior vertebras.

10. The spinal stabilization implant assembly of claim 6, wherein the intervertebral cage is attached to the back surface of the elongated body with a screw.

11. The spinal stabilization implant assembly of claim 1, wherein the top and bottom portions of the elongated body are pivotally connected to the central portion of the elongated body.

12. The spinal stabilization implant assembly of claim 11, further comprising top and bottom locking tabs configured to lock the angular positions of the top and bottom portions relative to the central portion.

13. The spinal stabilization implant assembly of claim 11, further comprising a keystone and wherein the keystone comprises top and bottom angled surfaces configured to lock the angular positions of the top and bottom portions relative to the central portion, when the keystone is attached to the central portion.

14. The spinal stabilization implant assembly of claim 1, wherein the cervical stabilization plate comprises an adjustable length.

15. The spinal stabilization implant assembly of claim 14, wherein the length of the cervical stabilization plate is adjusted via a ratchet mechanism.

16. The spinal stabilization implant assembly of claim 14, wherein the length of the cervical stabilization plate is adjusted by rotating a threaded rod.

17. The spinal stabilization implant assembly of claim 14, wherein the length of the cervical stabilization plate is adjusted via a cam mechanism.

18. The spinal stabilization implant assembly of claim 14, wherein the length of the cervical stabilization plate is adjusted via a side-sliding mechanism, and wherein the side-sliding mechanism comprises inserting plates of different height in a space between the top and central portions or the space between the bottom and central portions.

19. The spinal stabilization implant assembly of claim 14, wherein the length of the cervical stabilization plate is adjusted via a cam mechanism and wherein the cam mechanism comprises an oval shaped cam configured to be rotated in a space between the top and bottom portions.

20. The spinal stabilization implant assembly of claim 6, wherein the back surface of the elongated body comprises a recess and wherein the intervertebral cage is shaped and dimensioned to be inserted into the recess and to slidably engage the elongated body.

21. The spinal stabilization implant assembly of claim 6, wherein the elongated body comprises a metal and the intervertebral implant comprises PEEK.

22. The spinal stabilization assembly of claim 6, wherein the height of the protruding indent-tab matches the height of the intervertebral cage.

23. A spinal stabilization implant assembly comprising:
a first cervical stabilization plate comprising an elongated body having a top portion and a bottom portion;
a second cervical stabilization plate comprising an elongated body having a top portion and a bottom portion;
wherein the bottom portion of the first cervical stabilization plate is configured to be attached to a first vertebra and wherein the top portion of the second stabilization plate is configured to be stacked end-to-end below the bottom portion of the first cervical stabilization plate and to be attached to the same first vertebra.

24. The spinal stabilization assembly of claim 23, wherein the top portion of the first cervical stabilization plate is configured to be attached to a second vertebra, wherein the second vertebra is superior to the first vertebra, and wherein the bottom portion of the second stabilization plate is configured to be attached a third vertebra, wherein the third vertebra is inferior to the first vertebra.

25. A spinal stabilization method comprising:
providing a spinal stabilization implant assembly;
implanting the spinal stabilization implant assembly at least partially between a superior vertebra and an inferior vertebra;
wherein the spinal stabilization implant assembly comprises a cervical stabilization plate and one or more bone fasteners and wherein the cervical stabilization plate comprises an elongated body having left and right side surfaces, front and back surfaces and top and bottom surfaces;
wherein the elongated body comprises a central portion, a top portion and a bottom portion and wherein the top portion is bent at a first angle relative to the central portion and is dimensioned for capturing and fastening to a corner ridge of a vertebral wall of the superior vertebra and wherein the bottom portion is bent at a second angle relative to the central portion and wherein the second angle is opposite to the first angle and is dimensioned for capturing and fastening to a corner ridge of a vertebral wall of the inferior vertebra;
wherein the back surface comprises a protruding indent-tab, and wherein the protruding indent-tab is shaped and dimensioned to be implanted in an intervertebral space between the superior and the inferior vertebras; and
wherein the elongated body further comprises one or more through-openings extending from the front surface to the back surface and wherein the one or more bone fasteners are shaped and dimensioned to be inserted through the one or more through-openings, respectively, and to be attached to locations in said vertebral walls of the superior and inferior vertebras.

26. The spinal stabilization method of claim 25, further comprising inserting an intervertebral cage in the intervertebral space between the superior and inferior vertebras, and wherein the height of the protruding indent-tab matches the height of the intervertebral cage.

27. A spinal stabilization method comprising:
providing a first cervical stabilization plate comprising an elongated body having a top portion and a bottom portion;
providing a second cervical stabilization plate comprising an elongated body having a top portion and a bottom portion;
attaching the bottom portion of the first cervical stabilization plate to a first vertebra;
stacking the top portion of the second stabilization plate end-to-end below the bottom portion of the first cervical stabilization plate; and
attaching the top portion of the second stabilization plate to the same first vertebra.

28. The method of claim 27, further comprising attaching the top portion of the first cervical stabilization plate to a second vertebra, wherein the second vertebra is superior to the first vertebra, and attaching the bottom portion of the second stabilization plate to a third vertebra, wherein the third vertebra is inferior to the first vertebra.
